(19) United States
(12) Patent Application Publication
(10) Pub. No.: US 2025/0213607 A1
(43) Pub. Date: Jul. 3, 2025

(54) PHARMACEUTICAL COMPOSITIONS FOR IMPROVING INTESTINAL BACTERIAL FLORA AND RELATED TREATMENT METHODS

(71) Applicant: Murata Manufacturing Co., Ltd., Nagaokakyo-shi (JP)

(72) Inventors: Yuki KIMURA, Nagaokakyo-shi (JP); Daisuke Megumi, Nagaokakyo-shi (JP); Hajime Takase, Yokohama-shi (JP); Yusuke Kobayashi, Yokohama-shi (JP)

(21) Appl. No.: 19/016,307

(22) Filed: Jan. 10, 2025

(30) Foreign Application Priority Data
Jan. 12, 2024 (JP) 2024-003230

(51) Int. Cl. A61K 33/00; A61P 1/00; A61P 9/12; A61P 25/28

(52) U.S. Cl. CPC A61K 33/00; A61P 1/00; A61P 9/12; A61P 25/28

(57) ABSTRACT

The present disclosure provides novel pharmaceutical compositions, e.g., a pharmaceutical composition capable of improving the intestinal bacterial flora in vivo or increasing a short-chain fatty acid in vivo, which comprises MXene. Methods of treatment using such compositions are also provided.

### CROSS REFERENCE TO RELATED APPLICATION

The present application claims priority to Japanese Patent Application No. 2024-003230, filed Jan. 12, 2024, the entire contents of which are incorporated herein by reference.

### TECHNICAL FIELD

The present disclosure relates to a pharmaceutical composition, a method for improving an intestinal bacterial flora, a treatment method, and a prevention method.

### BACKGROUND ART

In recent years, the relevance between the intestinal environment and various diseases has been suggested, and the improvement of the intestinal environment is expected to have the treatment effect of these diseases.

JP 2023-12558A describes a pharmaceutical composition for improving the intestinal bacterial flora, containing 1-cyclopropyl-6 fluoro-1,4-dihydro-8 methyl-7-(2-amino-3-cyano-5-pyridyl)-4-oxo-3-quinoline carboxylic acid or a pharmaceutically acceptable salt thereof as an active ingredient.

Lifu Wang et al., “An engineered probiotic secreting Sj16 ameliorates colitis via Ruminococcaceae/butyrate/retinoic acid axis,” Bioengineering & Translational Medicine, Volume 6, Issue 3 (September 2021), describes that by administering genetically engineered E. coli to dextran sulfate sodium-induced colitis (DSS) mice as a model animal of inflammatory bowel disease (IBD), the diversity of intestinal bacterial flora is promoted to proliferate the number of Ruminococcaceae, and as a result, the production of Butyric acid can be enhanced.

V. Braniste et al., “The gut microbiota influences blood-brain barrier permeability in mice,” Science Translational Medicine, 6:263ra158 (2014), describes that diversity of intestinal bacteria can promote expression of Cldn and reduce the permeability of Blood Brain Barrier (BBB).

Luisa F. Gomez-Arango et al., “Increased Systolic and Diastolic Blood Pressure Is Associated With Altered Gut Microbiota Composition and Butyrate Production in Early Pregnancy,” Hypertension, Vol 68, Issue 4 (October 2016), describes that Butyrate can dominantly correct the hypertensive symptoms of a pregnant female.

### SUMMARY OF THE DISCLOSURE

### Problems to be Solved

JP 2023-12558A describes that an intestinal bacterial flora can be improved by administering a predetermined compound, and the research publications by Wang, Braniste, and Gomez-Arango, cited herein, describe that when E. coli is directly administered, it may proliferate Ruminococcaceae, a family of intestinal bacteria in the class Clostridia, and Butyric acid, which may be produced thereby, will contribute to the suppression of permeability enhancement in Blood Brain Barrier (BBB) and amelioration of hypertensive symptoms. However, there is no description about the action of MXene.

One object of the present disclosure is to provide a novel pharmaceutical composition, preferably a pharmaceutical composition capable of improving the intestinal bacterial flora in vivo or increasing a short-chain fatty acid in vivo.

### Solutions

The pharmaceutical composition of the present disclosure comprises MXene and is used for improving the intestinal bacterial flora in vivo.

### Effects

The present disclosure provides novel pharmaceutical compositions, including pharmaceutical compositions capable of improving the intestinal bacterial flora in vivo. In some aspects, such compositions may increase the amount or concentration of one or more short-chain fatty acids in vivo. The present disclosure may also provide treatment and preventative methods (e.g., based on the pharmaceutical compositions described herein).

In some aspects, the pharmaceutical composition of the present disclosure comprises MXene, can promote the proliferation of intestinal bacteria, can improve the intestinal bacterial flora, and/or can increase the amount or concentration of one or more short-chain fatty acids in vivo (e.g., in the blood or serum of a human subject). Therefore, in some aspects the present compositions may be useful for the treatment and/or prevention of various diseases.

Although not to be construed as being limited to a specific theory, the MXene used in pharmaceutical compositions of the present disclosure may promote the proliferation of intestinal bacteria in the intestine. In addition, the proliferation of one or more short-chain fatty acids, which can be produced by metabolism of these intestinal bacteria, is also expected to be promoted by the proliferation of these intestinal bacteria. As a result, the action of the short-chain fatty acid(s) is expected to promote the repair of the blood vessel barrier. The intake of MXene is also expected to lower the blood pressure. Since MXene is not considered to be absorbed from the intestinal tract, MXene is expected to pass through the gastrointestinal tract and be excreted as it is together with feces. As described above, although a living body originally has two excretion functions of bile excretion and urine excretion, the metabolic pathway of the pharmaceutical compositions described herein may also be interpreted as a third metabolic pathway, and is expected to lead to the reduction of treatment related to dialysis therapy in patients with renal failure, for example. In addition, MXene is also expected to adsorb disease-causing substances and the like, contained in the contents of the diet in the gastrointestinal tract and does not cause intestinal tract absorption.

### BRIEF DESCRIPTION OF DRAWINGS

FIGS. 1(a) and 1(b) are schematic cross-sectional views showing MXene particles of a layered material in one embodiment of the present disclosure, in which FIG. 1(a) shows single-layer MXene particles, and FIG. 1(b) shows multilayer (exemplarily, two layers) MXene particles.

FIG. 2 is a schematic cross-sectional view showing a material in one embodiment of the present disclosure.

FIGS. 3(a) and 3(b) show the results of a MXene administration test to a hypertensive mouse, in which FIG. 3(a) shows a change in systolic blood pressure (SBP), and FIG. 3(b) shows a change in diastolic blood pressure (DBP).

FIG. 4 shows a result of a MXene administration test to a hypertensive mouse, and shows a behavior area of a mouse in cognitive behavior analysis.

FIG. 5 shows a result of a MXene administration test to a hypertensive mouse, and shows a Time of Entries Discrimination Index in cognitive behavior analysis.

FIGS. 6(a) to 6(d) show the results of the MXene administration test to a hypertensive mouse, and in 16s-rRNA analysis, FIG. 6(a) shows the abundance ratio of family Eggerthellaceae in order Coriobacteriales of class Coriobacteriia of phylum Actinobacteriota;

FIG. 6(b) shows the abundance ratio of family Ruminococcaceae in order Oscillospirales of class Clostridia of phylum Firmicutes; FIG. 6(c) shows the abundance ratio of family Lachnospirales of class Clostridia of phylum Firmicutes of order Lachnospiraceae; and FIG. 6(d) shows the abundance ratio of family Butyricicoccaceae in order Oscillospirales of class Clostridia of phylum Firmicutes.

FIGS. 7(a) and 7(b) show the results of the MXene administration test to a hypertensive mouse, and in 16s-rRNA analysis, FIG. 7(a) shows a diversity of intestinal bacterial flora, and FIG. 7(b) shows R diversity drawn by PCoA.

FIGS. 8(a) to 8(c) show the results of a MXene administration test to a hypertensive mouse, and show changes in the amounts of FIG. 8(a) butyric acid, FIG. 8(b) acetic acid, and FIG. 8(c) propionic acid in CE-MS.

FIG. 9 shows the results of a test of MXene administration to a hypertensive mouse, and shows a change in Cldn5 in RNA seq of a callosum cell.

FIG. 10 shows the result of the MXene administration test to a hypertensive mouse, and shows a fluorescence microscopic observation image stained with myelin.

FIGS. 11(a) and 11(b) show the results of the MXene administration test to a hypertensive mouse, and show the amount of MBP measured by the WB method, where the MBP amount FIG. 11(a) is a photograph of the membrane after transfer, and FIG. 11(b) shows a ratio of the amount of MBP based on the amount of P-actin.

### DETAILED DESCRIPTION

The pharmaceutical composition of the present disclosure comprises Mxene. Mxene is typically a layered material having the form of one or more layers. In general, MXene has the form of particles of such a layered material (may comprise powders, flakes, nanosheets, etc.).

The Mxene preferably comprises two-dimensional particles of a layered material having one or more layers. In some aspects, at least one of the layers preferably comprises at least one metal selected from periodic table group 3, 4, 5, 6, and 7 metals, and at least one carbon atom or nitrogen atom.

The at least one group 3, 4, 5, 6, or 7 metal may comprise at least one metal selected from the group consisting of Ti, Zr, Hf, V, Nb, Ta, Cr, Mo, Sc, W, and Mn. In some aspects, the at least one metal is selected from the group consisting of Ti, V, Cr, and Mo.

In some aspects, the layer comprises a layer body represented by the following composition formula:

MₘXₙ
wherein “M” is at least one group 3, 4, 5, 6, and 7 metals,
   “X” is a carbon atom, a nitrogen atom, or a combination thereof,
   “n” is 1 to 4, and
   “m”>“n” and “m”≤5.

In some aspects, the layer further comprises a modification or termination “T” present on a surface of the layer body in which “T” is at least one selected from the group consisting of a hydroxyl group, a fluorine atom, a chlorine atom, an oxygen atom, and a hydrogen atom.

The pharmaceutical composition of the present disclosure comprises Mxene, e.g., two-dimensional particles having a layer body represented by MₘXₙ and T, and, in some aspects, can adsorb a substance that can cause a disease (disease causing substance), and thus is useful for the treatment and/or prevention of various diseases.

In the present disclosure, the layered material may be understood as a layered compound, and the layer is also referred to as “MₘXₙTₛ”. In the formula, “s” is an arbitrary number, and conventionally, “x” or “z” may be used instead of “s.” Hereinafter, the layered material may be referred to as MXene, the layer may be referred to as an MXene layer, and the two-dimensional particles may be referred to as MXene two-dimensional particles or MXene particles.

In the present disclosure, when an element is referred to as an “atom”, the oxidation number of the element is not limited to 0, and may be an arbitrary number within the range of possible oxidation numbers of the element.

In addition, with respect to the reference numerals of the general formulae shown in the present disclosure, unless otherwise specified, definitions for the same reference numerals are common among the general formulae including the reference numerals.

In the above formula: MₘXₙ, “m” may typically be, but not limited to, 2, 3, 4, or 5. Also, “n” may be, but is not limited to, 1, 2, 3, or 4. In one aspect, “m” may be 3 and “n” may be 2.

In the above formula: MₘXₙ, “M” may comprise, e.g., at least one metal selected from the group consisting of Ti, Zr, Hf, V, Nb, Ta, Cr, Mo, Sc, W, and Mn. In some aspects, “M” comprises at least one metal selected from the group consisting of Ti, V, Cr, and Mo.

The term MₘXₙ encompasses the following non-limiting set of exemplary compounds:
Sc₂C, Ti₂C, Ti₂N, Zr₂C, Zr₂N, Hf₂C, Hf₂N, V₂C, V₂N, Nb₂C, Ta₂C, Cr₂C, Cr₂N, Mo₂C, Mo_{1.3}C, Cr_{1.3}C, (Ti, V)₂C, (Ti, Nb)₂C, W₂C, W_{1.3}C, Mo₂N, Nb_{1.3}C, Mo_{1.3}Y_{0.6}C. (In the above formula, “1.3” and “0.6” mean about 1.3 (=4/3) and about 0.6 (=⅔), respectively);
Ti₃C₂, Ti₃N₂, Ti₃ (CN), Zr₃C₂, (Ti, V)₃C₂, (Ti₂Nb)C₂, (Ti₂Ta)C₂, (Ti₂Mn)C₂, Hf₃C₂, (Hf₂V)C₂, (Hf₂Mn)C₂, (V₂Ti)C₂, (Cr₂Ti)C₂, (Cr₂V)C₂, (Cr₂Nb)C₂, (Cr₂Ta)C₂, (Mo₂Sc)C₂, (Mo₂Ti)C₂, (Mo₂Zr)C₂, (Mo₂Hf)C₂, (Mo₂V)C₂, (Mo₂Nb)C₂, (Mo₂Ta)C₂, (W₂Ti)C₂, (W₂Zr)C₂, (W₂Hf)C₂,
Ti₄N₃, V₄C₃, Nb₄C₃, Ta₄C₃, (Ti, Nb)₄C₃, (Nb, Zr)₄C₃, (Ti₂Nb₂)C₃, (Ti₂Ta₂)C₃, (V₂Ti₂)C₃, (V₂Nb₂)C₃, (V₂Ta₂)C₃, (Nb₂Ta₂)C₃, (Cr₂Ti₂)C₃, (Cr₂V₂)C₃, (Cr₂Nb₂)C₃, (Cr₂Ta₂)C₃, (Mo₂Ti₂)C₃, (Mo₂Zr₂)C₃, (Mo₂Hf₂)C₃, (Mo₂V₂)C₃, (Mo₂Nb₂)C₃, (Mo₂Ta₂)C₃, (W₂Ti₂)C₃, (W₂Zr₂)C₃, (W₂Hf₂)C₃, (Mo_{2.7}V_{1.3})C₃. (In the above formula, “2.7” and “1.3” mean about 2.7 (=8/3) and about 1.3 (=4/3), respectively.)

Typically, in the above formula (MₘXₙ), “M” may be Ti or V, “X” may be a carbon atom or a nitrogen atom. In some aspects, “M” may be Ti, and “X” may be a carbon atom. In some aspects, MXene may be Ti₃C₂ Tₛ (i.e., where “M” is Ti, “X” is C, “n” is 2, and “m” is 3.). In this case, a precursor of such MXene (also referred to as a “MAX phase”) may be Ti₃AlC₂.

MXene can be produced by removing “A” atoms comprised in the MAX phase of the precursor. In some aspects, this MAX phase is represented by MₘAXₙ, where “M,” “m,” “X,” and “n” have the same meaning as described above, and “A” is at least one periodic table group 12, 13, 14, 15, or 16 element, and MXene may comprise such “A” atoms. In one aspect, the residual amount of “A” atoms comprised in MXene may be 10% by mass or less, e.g., 8% by mass or less, or 6% by mass or less with respect to the content of “A” atoms in the precursor.

In another aspect, the residual amount of “A” atoms may be more than 10% by mass. For example, those in which the “A” atom is removed from only a part of the MAX phase are also comprised in the technical scope of the MXene. Examples of such MXene include MXene in which “A” atoms are removed only from the vicinity of the end in the plane direction of the MAX phase (a direction parallel to the plane of the MₘXₙ layer comprised in the MAX phase). In this aspect, the residual amount of the “A” atoms may be, for example, 50% by mass or more, e.g., 80% by mass or more, or 90% by mass or more.

In some aspects, the content of lithium in the MXene may be 0% by mass to 0.1% by mass, e.g., 0% by mass to 0.01% by mass, or 0% by mass to 0.002% by mass. Without being bound to a theory, biocompatibility may be improved when the content of lithium is within the above range. The content of lithium in an MXene may be measured by inductively coupled plasma atomic emission spectrometry (ICP-AES).

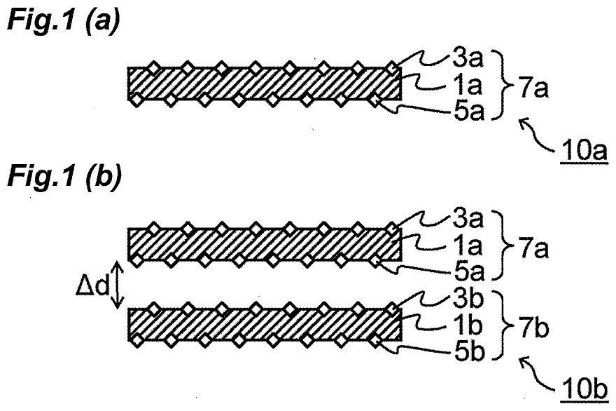

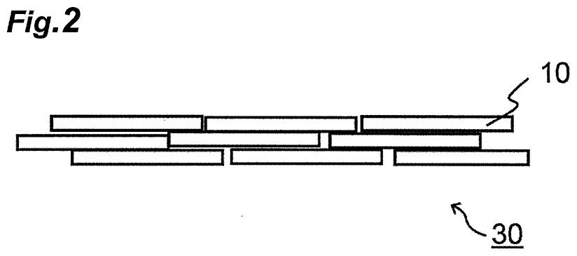

The MXene is an aggregate comprising MXene particles (hereinafter, simply referred to as “MXene particles”) 10a (single-layer MXene particles) of one layer schematically exemplified in FIG. 1(a). Typically, the MXene particle 10a is an MXene layer 7a having a layer body (MₘXₙ layer) 1a represented by MₘXₙ and one or more modifications or terminations T 3a, 5a may be present on the surface of the layer body 1a (more specifically, at least one of two surfaces facing each other in each layer). Therefore, the MXene layer 7a is also represented as “MₘXₙTₛ”, and “s” is an arbitrary number.

The MXene may comprise one or more layers. Examples of the MXene particles (multilayer MXene particles) of the plurality of layers include, but are not limited to, the MXene particle 10b of two layers as schematically shown in FIG. 1(b). 1b, 3b, 5b, and 7b in FIG. 1(b) are the same as 1a, 3a, 5a, and 7a in FIG. 1(a) described above. Two adjacent MXene layers (for example, 7a and 7b) of the multilayer MXene particles may not necessarily be completely separated from each other, but may be partially in contact with each other. In the single-layer MXene particle 10a, the multilayer MXene particle 10b are individually separated and exist in one layer. The MXene may comprise a mixture of the single-layer MXene particles 10a and the multilayer MXene particle 10b in which unseparated multilayer MXene particles 10b remain. Typically, at least one of the surfaces of the layer body 1a represented by MₘXₙ can be planar (two-dimensional), and all of the surfaces of the layer body 1a can be planar (two-dimensional).

Although the present embodiment is not limited, the thickness of each layer (corresponds to the MXene layers 7a and 7b) comprised in the MXene particles is, for example, 0.8 nm to 5 nm, e.g., 0.8 nm to 3 nm. The thickness may vary, e.g., depending on the number of M atom layers comprised in each layer. The thickness of each layer is determined as a number average dimension (for example, a number average of at least 40) based on an atomic force microscope (AFM) photograph or a transmission electron microscope (TEM) photograph.

For each laminate of MXene (particularly multilayer MXene particles that may be comprised), the interlayer distance may be, for example, 0.8 nm or more and 10 nm or less, particularly 0.8 nm to 5 nm, and more particularly about 1 nm, and the total number of layers may be 2 to 20,000. Alternatively, the void dimension is indicated by Ad in FIG. 1(b). The interlayer distance in MXene can be measured by obtaining an interplanar distance (the sum of the interlayer distance and the thickness of each layer) from the position of a peak corresponding to the (002) plane of MXene present at 2θ=100 (deg) or less in X-ray diffraction measurement of MXene and subtracting the thickness of each layer from the interplanar distance.

The MXene may comprise MXene particles having a small number of layers. The “small number of layers” means that, for example, the number of laminated MXene layers is six or less (e.g., 5, 4, 3, 2, or 1). In addition, the thickness of the multilayer MXene particles having a small number of layers in the lamination direction may be 15 nm or less, e.g., 10 nm or less (e.g., 15, 14, 13, 12, 11, 10, 9, 8, 7, 6, 5, 4, 3, 2, or 1 nm). Hereinafter, the “multilayer MXene particles having a small number of layers” may be referred to as “few-layer MXene particles.” The single-layer MXene particles and the few-layer MXene particles may be collectively referred to as “single-layer/few-layer MXene particles.”

In the MXene, the ratio of the single-layer/few-layer MXene particles having a thickness of 15 nm or less may be 0 vol % to 100 vol %, e.g., 0 vol % to 99 vol %, 0 vol % to 50 vol %, or 0 vol % to 30 vol %.

The major axis of MXene may be, e.g., 1 μm to 20 μm in a plane (hereinafter, also referred to as a “two-dimensional surface”) parallel to each layer. Hereinafter, the average value of the major axes of the two-dimensional surfaces may be referred to as “average flake size.”

Without being bound to a theory, the larger the average flake size is, the better the orientation of MXene is in the material comprising MXene. The average value of the major axes of the two-dimensional surfaces may be 1.5 μm or more, e.g., 2.5 μm or more. When the delamination treatment of MXene is performed by subjecting MXene to an ultrasonic treatment, most of MXene is reduced in a major axis to about several hundred nm by the ultrasonic treatment, and thus the film formed of the single-layer MXene delaminated by the ultrasonic treatment is considered to have low orientation of MXene.

The average value of the major axes of the two-dimensional surfaces is 20 μm or less, e.g., 15 μm or less, or 10 μm or less, from the viewpoint of dispersibility in the dispersion medium.

The major axis of the two-dimensional surface refers to a major axis when each MXene particle is approximated to an elliptical shape in an electron microscope photograph of MXene observed from a direction substantially orthogonal to a plane parallel to each layer, and the average value of the major axes of the two-dimensional surface refers to a number average of the major axes of 80 particles or more. As the electron microscope, a scanning electron microscope (SEM) photograph or a transmission electron microscope (TEM) photograph can be used.

The average value of the major axes of MXene of the present embodiment may be measured by dissolving a material comprising MXene in a solvent and dispersing the MXene in the solvent. Alternatively, it may be measured from an SEM image of the material.

The average value of the thickness of MXene in the present compositions may be 1 nm to 100 m, e.g., the thickness may be 50 μm or less, or 20 μm or less. On the other hand, in consideration of the thickness of the single-layer MXene particles, the lower limit of the thickness of MXene may be 1 nm.

The thickness of the MXene can be understood as a length in a direction substantially orthogonal to a plane parallel to each layer, and an average value of the thickness of the MXene is obtained as a number average dimension (for example, a number average of at least 40) based on an atomic force microscope (AFM) photograph or a transmission electron microscope (TEM) photograph.

MXene for use in the compositions and methods described herein may be produced by the following production method, but the present disclosure is not limited to MXene produced by this exemplary method.

In one aspect, the method for producing MXene comprises:
(a) preparing a precursor represented by the following formula:

MₘAXₙ
wherein “M” is at least one period table group 3, 4, 5, 6, and 7 metal,
   “X” is a carbon atom, a nitrogen atom, or a combination thereof,
   “A” is at least one group 12, 13, 14, 15, or 16 element,
   “n” is 1 to 4,
   “m”>“n” and “m”≤5;
(b) removing at least a portion of the “A” atoms from the precursor by etching using an etching solution to obtain the etched product; and
(c) cleaning the etched product to obtain a cleaned product,
and may further comprise:
(d) performing an intercalation treatment on the etching treatment product in a dispersion medium using a metal-containing compound to obtain an intercalated product; and (e) performing a delamination treatment on the intercalated product to obtain a delaminated product.

In one aspect, the etched product and the delaminated product may be used as the MXene, and preferably the cleaned product can be used as the MXene.

Each step of the aforementioned method is described in further detail below.

Step (a), preparation of a precursor represented by the formula MₘAXₙ, may comprise any combination of the following sub-steps and/or parameters.

First, a predetermined precursor is prepared. The predetermined precursor that can be used in the present embodiment is a MAX phase that is a precursor of MXene, and
represented by the following formula:

MₘAXₙ
wherein “M” is at least one group 3, 4, 5, 6, or 7 metal,
   “X” is a carbon atom, a nitrogen atom, or a combination thereof,
   “A” is at least one group 12, 13, 14, 15, or 16 element,
   “n” is 1 or more and 4 or less,
   “m”>“n” and “m”≤5.

“M,” “X,” “n,” and “m” have the same meaning as described above.

“A” is at least one group 12, 13, 14, 15, or 16 element, e.g., a group A element, or a group IIIA element and a group IVA element, and more particularly may comprise at least one element selected from the group consisting of Al, Ga, In, Tl, Si, Ge, Sn, Pb, P, As, S, and Cd (e.g., Al or Si).

The MAX phase has a crystal structure in which a layer constituted by “A” atoms is located between two layers represented by MₘXₙ (each “X” may have a crystal lattice located in an octahedral array of “M”). Typically, when m=n+1, the MAX phase comprises repeating units in which each one layer of X atoms is disposed in between adjacent layers of n+1 layers of “M” atoms (these are also collectively referred to as an “MₘXₙ layer”), and a layer of “A” atoms (“A atom layer”) is disposed as a layer next to the (n+1)th layer of M atoms. Such configurations may also be observed when m n+1.

The MAX phase may be produced by known methods. For example, a TiC powder, a Ti powder, and an Al powder may be mixed in a ball mill, and the resulting mixed powder may be fired under an Ar atmosphere to obtain a fired body (block-shaped MAX phase). Thereafter, the fired body obtained may be pulverized by an end mill to obtain a powdery MAX phase for the next step.

Step (b), removing at least a portion of the “A” atoms from the precursor by etching using an etching solution to obtain the etched product, may comprise any combination of the following sub-steps and/or parameters.

In step (b), an etching treatment for removing at least a part of the “A” atoms from the precursor (MAX phase) represented by MₘAXₙ is performed. As a result, an etched product in which at least a part of the layer composed of “A” atoms is removed is obtained while the MₘXₙ layer in the precursor is maintained. Conditions for the etching treatment are not particularly limited, and known conditions can be adopted. The etching may be performed using an etching solution comprising F⁻. Such an etching solution may comprise hydrofluoric acid, hydrochloric acid, phosphoric acid, or the like as an acid. In one aspect, examples of the etching solution comprise hydrofluoric acid; a mixed solution of hydrofluoric acid and hydrochloric acid; and a mixed solution of lithium fluoride and hydrochloric acid, and all of them may further comprise phosphoric acid. As the solvent in the etching solution, water may be used, and for example, pure water may be used.

The etching treatment may be performed as a slurry by mixing the precursor and the etching solution.

In some aspects, in step (b), the intercalation treatment may be performed simultaneously. By allowing a metal-containing compound to be described later to coexist in the etching solution, the etching treatment and the intercalation treatment can be simultaneously performed. In this case, step (e) described below may be further performed.

When the intercalation treatment is simultaneously performed in the step (b), the content of the metal-containing compound in the total of the precursor, the metal-containing compound, and the etching solution can be, for example, 0.001% by mass to 10% by mass, e.g., 0.01% by mass to 1% by mass, or 0.1% by mass to 1% by mass.

Step (c), cleaning the etched product to obtain a cleaned product, may comprise any combination of the following sub-steps and/or parameters.

In step (c), the treated product obtained by the etching treatment is cleaned to obtain a cleaned product. By performing the cleaning, the acid and the like used in the etching treatment can be sufficiently removed. The cleaning may preferably be carried out with water. The amount of water mixed with the etched product and the cleaning method are not particularly limited. For example, stirring, centrifugation, and the like may be performed by adding water. Examples of the stirring method include a stirring method using a handshake, an automatic shaker, a share mixer, a pot mill, or the like. The degree of stirring such as the stirring speed and the stirring time may be adjusted according to the amount, concentration, and the like of the etched product to be treated. The cleaning with water may be performed once or more, and cleaning with water is preferably performed a plurality of times. For example, specifically, the cleaning with water may be performed by sequentially performing step (i) (to the treated product or the remaining precipitate obtained in the following (iii)), adding water and stirring, step (ii), centrifuging the stirred product, and step (iii) discarding the supernatant after centrifugation, and the steps (i) to (iii) may be repeated within a range of 2 times or more, for example, 15 times or less.

Step (d), performing an intercalation treatment on the etching treatment product in a dispersion medium using a metal-containing compound to obtain an intercalated product, may comprise any combination of the following sub-steps and/or parameters.

In step (d), an intercalation treatment for obtaining an intercalated product is performed by performing an intercalation treatment on the etched product in a dispersion medium using a metal-containing compound comprising a metal ion. As a result, an intercalated product in which a metal ion comprised in the metal-containing compound is intercalated between two adjacent MₘXₙ layers is obtained. The metal ion may comprise a monovalent metal ion, and examples of the monovalent metal ion include alkali metal ions such as a lithium ion, a sodium ion, and a potassium ion, a copper ion, a silver ion, and a gold ion.

Examples of the metal-containing compound include an ionic compound in which the metal ion and the anion are bonded. Examples of the ionic compounds include a sulfide salt including an iodide, a phosphate, and a sulfate, a nitrate, an acetate, and a carboxylate of the above metal ion. As the metal ion, a lithium ion is preferable, and as the metal-containing compound, a metal-containing compound comprising a lithium ion is preferable, an ionic compound of a lithium ion is more preferable, and one or more of an iodide, a phosphate, and a sulfide salt of a lithium ion is further preferable. When a lithium ion is used as the metal ion, water hydrated to the lithium ion is considered to have the most negative dielectric constant, and thus it is easy to form a monolayer.

The content of the metal-containing compound in the total of the etched product, the metal-containing compound, and the dispersion medium can be, for example, 0.001% by mass to 10% by mass, e.g., 0.01% by mass to 1% by mass, or 0.1% by mass to 1% by mass. Dispersibility in a dispersion medium is typically good when the content of the metal-containing compound is within the above range.

A specific method of the intercalation treatment is not particularly limited, and for example, the dispersion medium, the etched product, and the metal-containing compound may be mixed and stirred, or may be left to stand. For example, stirring at room temperature can be mentioned. Examples of the stirring method include a method using a stirring bar such as a stirrer, a method using a stirring blade, a method using a mixer, a method using a centrifugal device, and the like, and the stirring time can be set according to the production scale of the single-layer/few-layer MXene particles, and can be set, for example, for 12 to 24 hours. The order of mixing the dispersion medium, the etched product, and the metal-containing compound is not particularly limited, but in one aspect, the dispersion medium and the etched product may be mixed, and then the metal-containing compound may be mixed. Typically, the etching solution after the etching treatment may be used as the dispersion medium.

Step (e), performing a delamination treatment on the intercalated product to obtain a delaminated product, may comprise any combination of the following sub-steps and/or parameters.

In step (e), the intercalated product obtained by performing the intercalation treatment is subjected to a delamination treatment to obtain a delaminated product. The delamination treatment comprises peeling at least a part between two adjacent MₘXₙ layers by applying a shear stress to the intercalated product. By the delamination treatment, the MXene particles can be formed into a single layer or a small layer.

Conditions for the delamination treatment are not particularly limited, and the delamination treatment can be performed by a known method. For example, as a method of applying a shear stress to the intercalated product, there is a method of dispersing the intercalated product in a dispersion medium and stirring the dispersion medium. Examples of the stirring method include ultrasonic treatment, handshaking, and stirring using an automatic shaker. The degree of stirring such as the stirring speed and the stirring time may be adjusted according to the amount, concentration, and the like of the treated product to be treated. For example, the slurry after the intercalation is centrifuged to discard the supernatant liquid, then pure water is added to the remaining precipitate, and stirring is performed by, for example, a handshake or an automatic shaker to perform layer separation. The removal of the unpeeled substance comprises a step of performing centrifugal separation to discard the supernatant, and then cleaning the remaining precipitate with water. For example, (i) pure water is added to the remaining precipitate after discarding the supernatant and stirred, (ii) centrifugation is performed, and (iii) the supernatant is recovered. This operation of (i) to (iii) is repeated 1 time or more, preferably 2 times or more and 10 times or less to obtain a supernatant comprising single-layer/few-layer MXene particles as a delaminated product. Alternatively, the supernatant may be centrifuged, and the supernatant after centrifugation may be discarded to obtain a clay comprising single-layer/few-layer MXene particles as a delaminated product.

In the method for producing MXene, when the intercalation treatment is performed, the cleaning treatment may be further performed at an arbitrary stage after the intercalation treatment, preferably at a stage after the delamination treatment. By performing such a cleaning treatment, the metal ion and the metal-containing compound used for the intercalation can be sufficiently removed. Typically, such a cleaning treatment is performed after the step (e).

In one aspect, the cleaning treatment after the intercalation treatment may be performed in the same manner as in the step (c). In another aspect, after the delaminated product is acid-treated, the acid-treated product may be cleaned in the same manner as in the step (c). In these cases, the etched product in the step (c) may be replaced with a delaminated product or an acid-treated product, and the cleaning treatment may be performed.

The acid treatment can be performed by mixing and stirring the delaminated product and the acid solution. Examples of the acid include inorganic acids such as hydrochloric acid, sulfuric acid, nitric acid, phosphoric acid, perchloric acid, hydroiodic acid, hydrobromic acid, and hydrofluoric acid; an organic acid such as acetic acid, citric acid, oxalic acid, benzoic acid, or sorbic acid may be appropriately used, and the concentration of the acid in the acid solution may be appropriately adjusted according to the delaminated product. The stirring can be performed using a handshake, an automatic shaker, a share mixer, a pot mill, or the like. The acid treatment may be performed one or more times, and if necessary, an operation of mixing with a fresh acid solution (acid solution not used for the acid treatment) and stirring may be performed within a range of two or more times, for example, 10 times or less.

The intermediate and the target product in the production method described above may be isolated by a commonly used purification method. Examples the purification method includes suction filtration; and drying such as heat drying, freeze drying, and vacuum drying.

### Pharmaceutical Compositions and Methods of Treatment

The MXene compositions described herein may improve the intestinal bacterial flora of a human subject in vivo. In particular, the present MXene compositions may promote growth of intestinal bacteria in vivo. Such intestinal bacteria may preferably comprise intestinal bacteria that increase the amount or concentration of one or more short-chain fatty acids in the human subject (e.g., as measured in a whole blood or plasma sample obtained from the subject). Intestinal bacteria that increase a short-chain fatty acid preferably comprise those of class Clostridia of phylum Firmicutes, more preferably those in family Eggerthellaceae of order Coriobacteriales of class Coriobacteriia of phylum Actinobacteriota, those in family Ruminococcaceae of order Oscillospirales of class Clostridia of phylum Firmicutes, those in family Lachnospiraceae of order Lachnospirales of class Clostridia of phylum Firmicutes, and even more preferably those in family Butyricicoccaceae of order Oscillospirales of class Clostridia of phylum Firmicutes.

Examples of short-chain fatty acids within the scope of the present disclosure include fatty acids having 1 to 4 carbon atoms, and preferably include butyric acid, acetic acid, and propionic acid. The short-chain fatty acid may have an action of repairing a blood vessel barrier. Such a blood vessel barrier comprises, without limitation, the intestinal barrier and the blood-brain barrier.

In addition, the MXene compositions described herein may be used to lower blood pressure in vivo in a subject in need thereof (e.g., by promoting vascular health as a result of reparations to one or more of the subject's blood vessel barriers).

Although the present disclosure should not be construed as being limited to a particular theory, the short-chain fatty acids produced following treatment with the MXene compositions described herein are believed to display three mechanisms of action: (i) repairing a blood vessel barrier and/or suppressing excessive enhancement of permeability; (ii) enhancing anti-inflammatory action; and (iii) ameliorating hypertension.

Regarding the above point (i), a short-chain fatty acid contributes to maintenance of homeostasis of blood vessel barriers (in particular, the blood-brain barrier), suppresses excessive enhancement of permeability, and can act protectively on brain tissue. This functionality can be confirmed by enhancement of Cldn, which is an RNA encoding Claudin, a protein found in blood vessel barriers (and in particular, the blood-brain barrier). Such blood vessel barriers include both the intestinal barrier and the blood-brain barrier.

Regarding the above point (ii), a short-chain fatty acid is an action that acts as a ligand of a G-protein coupled receptor (GPR) and has an anti-inflammatory action via the immune system in the whole body. In the central nervous system, among a short-chain fatty acid, the permeation efficiency of the blood-brain barrier is particularly high, and has an anti-inflammatory action due to the inhibitory action of Histone Deacetylase (HDAC) activated at the injury site and the like. The action of butyric acid on the cerebral white matter can be confirmed, for example, from the fact that the GPR signaling pathway of the cerebral white matter is significantly enriched by MFT administration in RNA-sequencing data (functional enrichment analysis using Database for Annotation, Visualization and Integrated Discovery (DAVID)) of a hypertensive mouse.

Regarding the above point (iii), a short-chain fatty acid may have an action of lowering blood pressure. Cerebral white matter (neural axons and myelin [complementary]) is a known fact to be damaged in hypertension. Such a hypotensive action can be confirmed from the viewpoint that a protective action against the central component (myelin) of the white matter is confirmed in the cerebral white matter of a hypertensive mouse, and the blood pressure decreases.

In view of the above, MXene of the present disclosure may be administered to a subject to promote any combination of the following actions and/or outcomes (i) repairing a blood vessel barrier and/or suppressing excessive enhancement of permeability; (ii) enhancing anti-inflammatory action; and (iii) ameliorating hypertension. Ameliorating hypertension also comprises lowering of blood pressure in vivo.

The MXene can be used for treatment or prevention of a disease in which improvement of symptoms can be expected by repair of a blood vessel barrier in vivo. Examples of such diseases include: vascular dementia, Parkinson's disease, inflammatory bowel disease, chronic renal failure, irritable bowel syndrome, and ischemic and demyelinating central nervous system disease. Thus, in some aspects the disclosure provides a methods of treating any of the foregoing diseases, comprising administering an MXene composition as described herein, to a subject in need thereof, in an amount effect to reduce one or more symptoms of the disease. Administration may be performed 1, 2, 3, 4, or 5 times daily, per week, per month, etc., using any dosage form described herein. In some aspects, the MXene is present in an amount of 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18. 19, 20, 21, 22, 23, 24, 25, 26, 27, 28. 29, 30, 31, 32, 33, 34, 35, 36, 37, 38. 39, 40, 41, 42, 43, 44, 45, 46, 47, 48. 49, 50, 51, 52, 53, 54, 55, 56, 57, 58. 59, 60, 61, 62, 63, 64, 65, 66, 67, 68. 69, 70, 71, 72, 73, 74, 75, 76, 77, 78. 79, 80, 81, 82, 83, 84, 85, 86, 87, 88. 89, 90, 91, 92, 93, 94, 95, 96, 97, 98. 99, 100 mg, or in an amount within a range defined by a pair of endpoints selected from any of the foregoing amounts (which are reflected in mg).

The MXene and the pharmaceutical composition can be administered orally.

The pharmaceutical composition according to the present embodiment can be in various dosage forms depending on usage. Examples of such a dosage form include powder, a granule, a fine granule, a dry syrup, a tablet, a capsule, liquid, and a sublingual agent, and also include an injection, an ointment, a suppository, and a patch. In one embodiment, a composition is administered in solid, semi-solid, micro-emulsion, gel, or liquid form. Examples of such dosage forms include tablet forms disclosed in U.S. Pat. Nos. 3,048,526, 3,108,046, 4,786,505, 4,919,939, and 4,950,484; gel forms disclosed in U.S. Pat. Nos. 4,904,479, 6,482,435, 6,572,871, and 5,013,726; capsule forms disclosed in U.S. Pat. Nos. 4,800,083, 4,532,126, 4,935,243, and 6,258,380; or liquid forms disclosed in U.S. Pat. Nos. 4,625,494, 4,478,822, and 5,610,184; each of which is incorporated herein by reference with respect to the disclosure of compositions and parameters for pharmaceutical dosage forms and methods of manufacturing the same. MXene pharmaceutical compositions may also be formulated as a modified release dosage form, including immediate-, delayed-, extended-, prolonged-, sustained-, pulsatile-, controlled-, extended, accelerated-, fast-, targeted-, programmed-release, and gastric retention dosage forms. These dosage forms can be prepared according to known methods and techniques (see, Remington: The Science and Practice of Pharmacy, 23^{rd} Ed. (Edited by Adeboye Adejare, Academic Press, 2020 (“Remington”); Modified-Release Drug Deliver Technology, Rathbone et al., Eds., Drugs and the Pharmaceutical Science, Marcel Dekker, Inc.: New York, N.Y, 2002; Vol. 126, which are each herein incorporated by reference in their respective entirety).

The pharmaceutical composition according to the present embodiment can be a pharmaceutical composition comprising MXene as an active ingredient and further at least one pharmaceutically acceptable additive. Such pharmaceutically acceptable additives may be made by known methods and selected according to the dosage form. Examples of such additives include an excipient, a disintegrant, a binder, a lubricant, a diluent, a buffering agent, an isotonizing agent, a preservative, a wetting agent, an emulsifying agent, a dispersing agent, a stabilizing agent, and a solubilizing agent. The pharmaceutical composition of the present disclosure can be prepared by appropriately mixing the MXene and the additive or diluting and dissolving the MXene with an additive. For example, the pharmaceutically acceptable additive may comprise one or more, or any combination of, the excipients, disintegrants, binders, lubricants, diluents, buffering agents, isotonizing agents, preservatives, wetting agents, emulsifying agent, dispersing agents, stabilizing agents, and solubilizing agents described in Remington, supra. In some aspects, the pharmaceutical composition comprises at least one MXene compound described herein, and at least one carrier, disintegrant, diluent, binder, or glidant, optionally in an oral dosage form (e.g., as a tablet).

The pharmaceutical composition according to the present embodiment can be administered systemically or locally, orally or parenterally (nasal, pulmonary, intravenous, enteral, subcutaneous, muscle, percutaneous). In one aspect, the pharmaceutical composition according to the present embodiment can be administered orally.

When the pharmaceutical composition of the present disclosure is used for treatment, the dose or effective amount of MXene as an active ingredient thereof is appropriately determined depending on the age, sex, weight, disease, degree of treatment, and the like of the patient. For example, in the case of oral administration, the dose may be appropriately administered once or in several divided doses in the range of about 100 mg to 10 g/body per day of an adult (body weight of 60 kg) as an effective amount. In some cases, the effective amount of the MXene compound in a pharmaceutical composition used in the methods described herein corresponds to about 50 to 1,000 mg of compound per adult subject, such as 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500, 550 mg, 600 mg, 650 mg, 700 mg, 750 mg, 800 mg, 850 mg, 900 mg, 950 mg or 1000 mg, or an amount within a range defined by a pair of endpoints selected from any of the foregoing amounts. In some aspects, the effective amount of the MXene compound in a pharmaceutical composition used in the methods described herein corresponds to about 1, 2, 3, 4, 5, 6, 7, 8, 9, or 10 g, or an amount within a range defined by a pair of endpoints selected from any of the foregoing amounts.

In addition, the pharmaceutical composition comprising MXene can be used for producing a medicine for treating or preventing a disease.

### EXAMPLES

The present disclosure will be described more specifically with reference to the following examples, but the present disclosure is not limited thereto.

### Example 1. Production of MXene

In Examples 1 and 2, (1) preparation of a precursor (MAX), (2) etching of the precursor, and (3) cleaning and drying described in detail below were sequentially performed to produce MXene two-dimensional particles.

### (1) Production of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was calcined in an Ar atmosphere at 1,350° C. for 2 hours. The fired body (block) thus obtained was crushed with an end mill to a maximum size of 40 μm or less. In this way, Ti₃AlC₂ particles were obtained as a precursor (MAX).

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.

### (Etching Conditions)

Precursor: Ti₃AlC₂ (Sieving with 45 μm mesh)
Etching solution composition: 50% by mass HF 6 mL, H₂O 18 mL
HCl (12M) 36 mL
Precursor charge amount: 3.0 g
Etching container: 100 mL Aiboy (wide-mouth bottle)
Etching temperature: 35° C.
Etching time: 24 h
Stirrer rotation speed: 400 rpm

### (3) Cleaning and Drying

The slurry was divided into two portions, each of which was inserted into two 50 mL centrifuge tubes, centrifuged under the condition of 3,500G using a centrifuge, and then the supernatant was discarded. An operation of adding 40 mL of pure water to each centrifuge tube, centrifuging again at 3,500 G, and separating and removing the supernatant was repeated 11 times. After final centrifugation, the supernatant was discarded to obtain a Ti₃C₂ Tₛ-water medium clay. The obtained clay was dried by freeze drying to obtain dry powder of MXene.

### Animal Experiment 1. Administration Experiment to Hypertensive Mice

Hypertension causes brain capillaries to become inflamed and produce chronic hypoperfusion. This may result in lower cognitive function and in extreme cases, vascular dementia. In Aanimal Experiment 2, MXene was administered to a mouse having genetically hypertension to remove inflammatory substances in the body, and it was confirmed whether recovery of cognitive function was expected. The administration period was 56 days, and the dose was 8% of the food consumption. After the end of the administration period, blood pressure fluctuation and cognitive behavior analysis (Novel Object Recognition Test) were performed. As a comparative example, a control group to which MXene was not administered was also prepared.

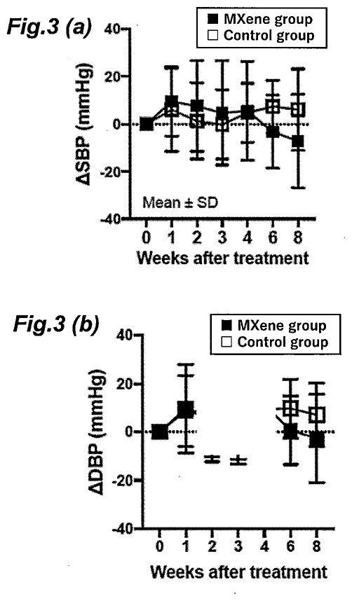

Changes in systolic blood pressure (SBP) and diastolic blood pressure (DBP) in the MXene group and the control group were measured. Comparison was performed by taking a difference based on the blood pressure value on the first day. The results are shown in FIGS. 3(a) and 3(b). A decrease in blood pressure was observed in the MXene group around the fourth week.

In the behavior analysis (Novel Object Recognition Test), a black box having a vertical width of 400 mm, a horizontal width of 400 mm, and a height of 400 mm was used. On days 1 and 2, mice were left in the box for 10 minutes to acclimatize. On day 3, two identical substances (old substances) were placed in cages, and the mice were left for 10 minutes. On day 4, one of the objects was changed to anew substance, and the mice were left for 10 minutes. Time for searching both objects was measured, and the ratio of the search time for a new substance to the total search time (Times of Entries Discrimination Index) was calculated according to the following equation:

$$Times of Entries Discrimination Index = [ n / ( n + f ) ] - 0.5$$

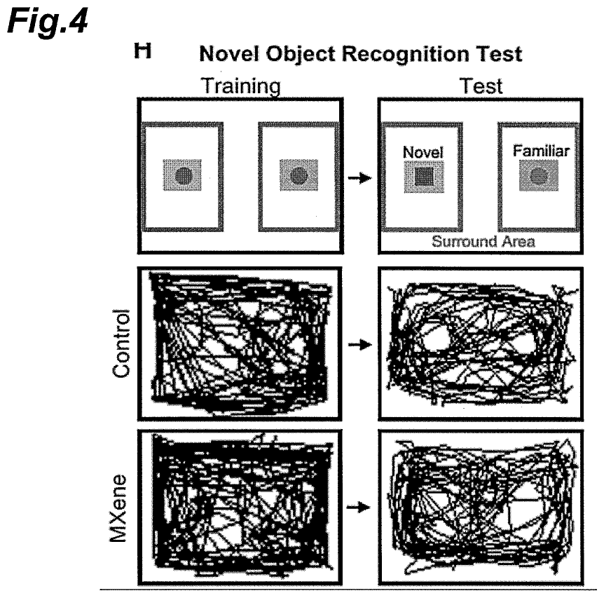

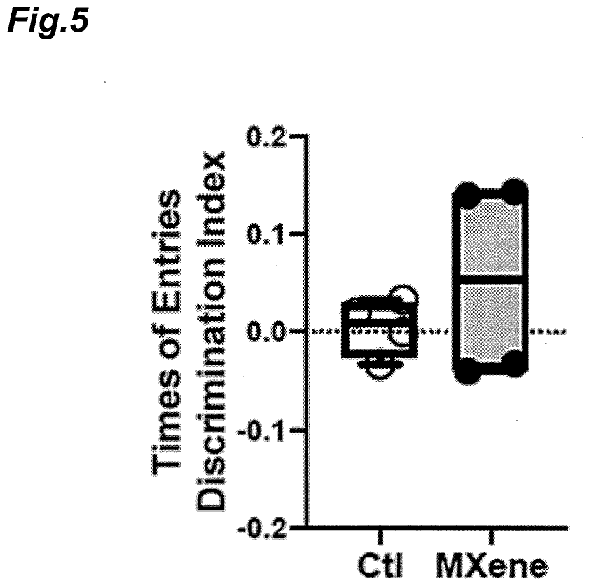

In this formula, “n” represents the number of times of touching the new object, and “f” represents the number of times of touching the conventional object. This formula was used as an index of memory learning ability. The mouse action region is shown in FIG. 4, and the Times of Entries Discrimination Index is shown in FIG. 5.

As shown in FIG. 5, in the Novel Object Recognition Test, the Times of Entries Discrimination Index was higher in the MXene group, and was a positive number. This indicates that the number of times of interest in and approaching the new substance is higher than that of the old substance. That is, it suggests that mouses recognized a new substance as something new, and that their cognitive function has recovered.

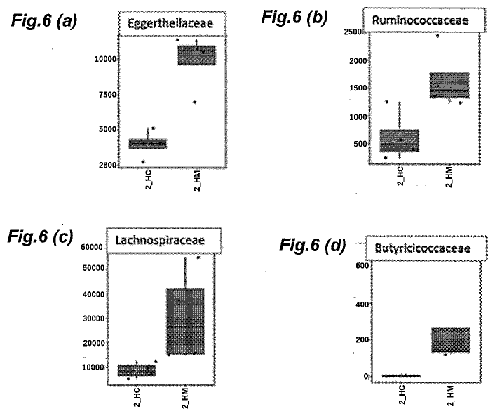

As shown in FIGS. 6(a) to 6(d), the abundance ratio of the intestinal bacterial flora of the mouse was compared between the groups by 16s-rRNA analysis. It was confirmed that those in family Eggerthellaceae of order Coriobacteriales of class Coriobacteriia of phylum Actinobacteriota, those in family Ruminococcaceae of order Oscillospirales of class Clostridia of phylum Firmicutes, those in family Lachnospiraceae of order Lachnospirales of class Clostridia of phylum Firmicutes, or those in family Butyricicoccaceae of order Oscillospirales of class Clostridia of phylum Firmicutes have proliferated.

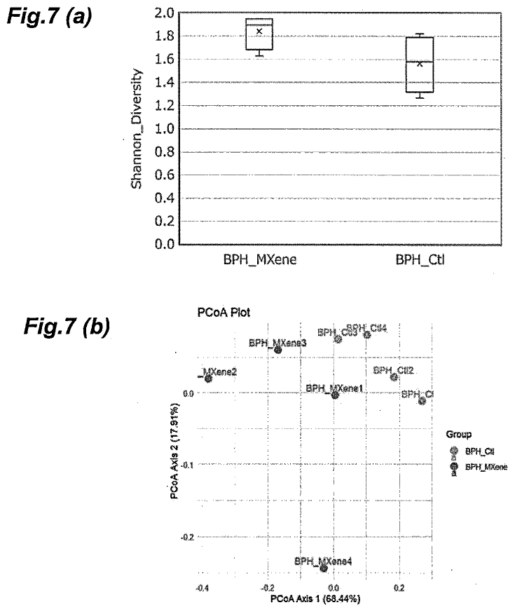

As shown in FIGS. 7(a) and 7(b), the diversity of the intestinal bacterial flora of the mouse obtained by 16s-rRNA analysis was compared. The a diversity was visualized using the Shannon Index, and the R diversity was visualized by PCoA (Principal Coordinates Analysis) after analysis based on the Bray-Curtis distance. It was confirmed that the diversity of the bacterial flora was changed between both groups by the administration of MXene.

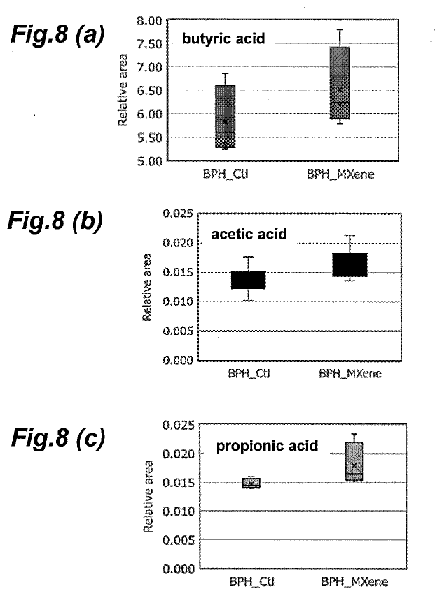

A metabolome analysis using CE-MS (capillary electrophoresis mass spectrometer) was performed on mouse serum, and changes in living body components by MXene were confirmed. As shown in FIGS. 8(a) to 8(c), the proliferation of butyric acid, acetic acid, and propionic acid as a short-chain fatty acid was confirmed in the MXene group.

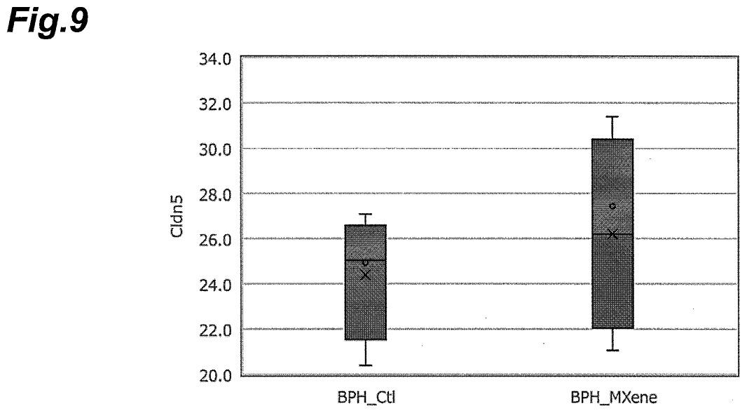

RNA_seq of a callosum cell was performed using a next generation sequencer. As shown in FIG. 9, proliferation of Cldn5, which is a gene that encodes a protein that forms the blood-brain barrier (Claudin), was confirmed.

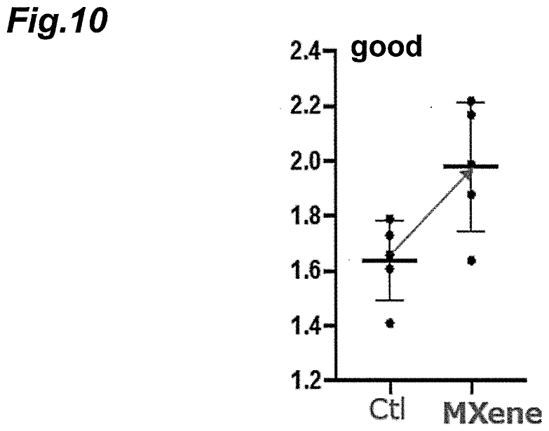

A Callosum staining evaluation of specimens was performed. The degree of damage can be confirmed by staining myelin that functionalizes nerve axons. The more myelin remains, the greater the brightness at the time of dyeing, and it can be determined that the damage is small. For image analysis, in order to correct variations at the time of imaging, the brightness of a white matter part based on a part called cortex was compared. As shown in FIG. 10, it was confirmed that the brightness was significantly higher and white matter damage was suppressed in the MXene group.

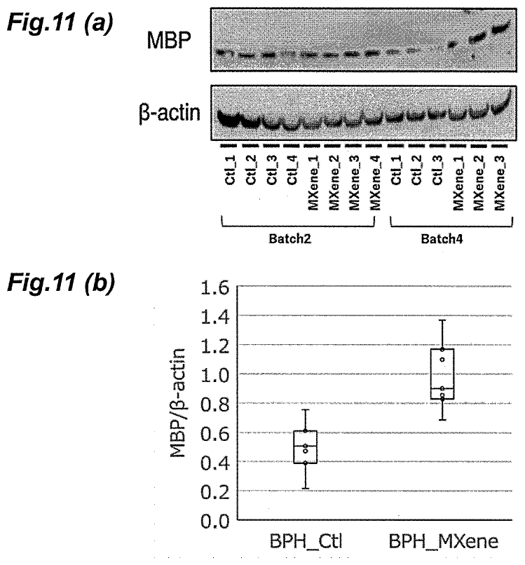

In response to the above results, WB measurement of MBP was performed in order to measure the amount of myelin. The method was a general WB method, in which a protein was extracted from a tissue of callosum, transferred to a membrane after gel electrophoresis, and a band was detected by an antibody reaction. In the measurement, the amount of MBP based on the amount of β-actin as the total amount of protein was compared among the specimens. Quantification by image processing was performed based on the marker floating on the membrane. As shown in FIGS. 11(a) and 11(b), an increase in MBP was confirmed in the MXene administration group.

It was observed that MXene increases the short-chain fatty acid-producing bacteria, those in family Eggerthellaceae of order Coriobacteriales of class Coriobacteriia of phylum Actinobacteriota, those in family Ruminococcaceae of order Oscillospirales of class Clostridia of phylum Firmicutes, those in family Lachnospiraceae of order Lachnospirales of class Clostridia of phylum Firmicutes, or those in family Butyricicoccaceae of order Oscillospirales of class Clostridia of phylum Firmicutes in the intestine. The proliferated short-chain fatty acid is considered to have promoted the decrease in blood pressure and the repair of BloodBrainBarrier, and contributed to the improvement of the cognitive function.

Furthermore, in Examples, it was confirmed that MXene conditions intestinal bacteria and proliferates short-chain fatty acid (SCFAs)-producing bacteria, particularly butyric acid-producing bacteria. Here, there are roughly three conceivable actions of SCFAs (particularly butyric acid).

The first is to contribute to the maintenance of homeostasis of the cerebral blood vessel endothelial cell/blood-brain barrier, suppress excessive enhancement of permeability, and act on brain tissue in a protective manner. This can be confirmed by enhancement of Cldn5 that is an RNA encoding Claudin that is a protein forming the blood-brain barrier.

The second is an action that acts as a ligand of a G-protein coupled receptor (GPR) and has an anti-inflammatory action via the immune system in the whole body. In the central nervous system, among a short-chain fatty acid, the permeation efficiency of the blood-brain barrier is particularly high, and has an anti-inflammatory action due to the inhibitory action of Histone Deacetylase (HDAC) activated at the injury site and the like. In fact, in the above Examples, also in the RNA-sequencing data (functional enrichment analysis using Database for Annotation, Visualization and Integrated Discovery (DAVID)) of the hypertensive mouse, the GPR signaling pathway of the cerebral white matter was observed to have been significantly enriched by MFT administration, and butyric acid acts on the cerebral white matter.

The third is an action of lowering blood pressure. Cerebral white matter (neural axons and myelin (complementary)) is a known fact to be damaged in hypertension. Therefore, in the cerebral white matter of a hypertensive mouse, it is supposedly confirmed that Mxene have a hypotensive action based on the observed actions; the protective action on the central component (myelin) of the white matter and the lowered blood pressure.

From the above points, it was confirmed that MXene has a multi-target protective action against damaged white matter by conditioning intestinal bacteria and proliferating short-chain fatty acid (SCFAs)-producing bacteria, particularly butyric acid-producing bacteria.

The present disclosure comprises the following:
[1] A pharmaceutical composition comprising MXene for improving intestinal bacterial flora in vivo or increasing a short-chain fatty acid in vivo.
[2] The pharmaceutical composition according to [1], wherein
   the Mxene comprises two-dimensional particles having one or more layers, and
   the layer comprises at least one metal selected from group 3, 4, 5, 6, and 7 metals and at least one selected from a carbon atom and a nitrogen atom.
[3] The pharmaceutical composition according to [2], wherein the layer comprises a layer body represented by the following formula:

MₘXₙ
wherein M is at least one group 3, 4, 5, 6, and 7 metals,
   X is a carbon atom, a nitrogen atom, or a combination thereof,
   n is 1 to 4, and
   m is more than n and 5 or less.
[4] The pharmaceutical composition according to [3], wherein the layer further comprises a modification or termination T present on a surface of the layer body in which T is at least one selected from the group consisting of a hydroxyl group, a fluorine atom, a chlorine atom, an oxygen atom, and a hydrogen atom.
[5] The pharmaceutical composition according to any one of [1] to [4], wherein the Mxene comprises Ti₃C₂.
[6] The pharmaceutical composition according to any one of [1] to [5], wherein the improving intestinal bacterial flora comprises proliferating intestinal bacteria that increase a short-chain fatty acid.
[7] The pharmaceutical composition according to any one of [1] to [6], wherein intestinal bacteria metabolizing the short-chain fatty acid comprise those in class Clostridia of phylum Firmicutes.
[8] The pharmaceutical composition according to any one of [1] to [7], wherein intestinal bacteria metabolizing the short-chain fatty acid comprise those in family Eggerthellaceae of order Coriobacteriales of class Coriobacteriia of phylum Actinobacteriota, those in family Ruminococcaceae of order Oscillospirales of class Clostridia of phylum Firmicutes, those in family Lachnospiraceae of order Lachnospirales of class Clostridia of phylum Firmicutes, or those in family Butyricicoccaceae of order Oscillospirales of class Clostridia of phylum Firmicutes.
[9] The pharmaceutical composition according to any one of [1] to [8], wherein the short-chain fatty acid comprises butyric acid.
[10] The pharmaceutical composition according to any one of [1] to [9], which is further used for one or two or more selected from the following (i) to (iii):
   (i) repairing a blood vessel barrier and/or suppressing excessive enhancement of permeability;
   (ii) enhancing anti-inflammatory action; and
   (iii) ameliorating hypertension.
[11] The pharmaceutical composition according to any one of [1] to [10], wherein the blood vessel barrier is an intestinal barrier or a blood-brain barrier.
[12] The pharmaceutical composition according to any one of [1] to [11], which is further used for lowering blood pressure.
[13] The pharmaceutical composition according to any one of [1] to [12], which is for oral administration.
[14] A method for improving intestinal bacterial flora or increasing a short-chain fatty acid in vivo, comprising administering an effective amount of MXene to a subject.
[15] The method according to [14], wherein the MXene is administered orally.
[16] A method for treating or preventing a disease in which improvement of symptoms can be expected by repair of a blood vessel barrier, the method comprising administering an effective amount of MXene to a subject.
[17] The method according to [16], wherein the disease comprises one or more selected from vascular dementia, Parkinson's disease, inflammatory bowel disease, chronic renal failure, irritable bowel syndrome, and ischemic and demyelinating central nervous system disease.
[18] The method for treating or preventing according to [16], wherein the MXene is administered orally.

### REFERENCE SIGNS LIST

1a, 1b Layer body (MₘXₙ layer)
3a, 5a, 3b, 5b Modifier or terminal T
7a, 7b MXene layer
10, 10a, 10b MXene particles (two-dimensional particles of layered material)

What is claimed is:

1. A pharmaceutical composition comprising MXene and one or more pharmaceutically acceptable additives, wherein the MXene is present in an amount effective for improving intestinal bacterial flora in vivo or increasing an amount or concentration of one or more short-chain fatty acids in vivo in a human subject.

2. The pharmaceutical composition according to claim 1, wherein
the Mxene comprises two-dimensional particles having one or more layers, and
at least one of the one or more layers comprises at least one group 3, 4, 5, 6, or 7 metal, and at least one carbon atom and/or nitrogen atom.

3. The pharmaceutical composition according to claim 2, wherein the at least one of the one or more layers comprises a layer body represented by the following formula:
MₘXₙ
wherein “M” is at least one group 3, 4, 5, 6, or 7 metal,
“X is a carbon atom, a nitrogen atom, or a combination thereof,
“n” is 1 to 4, and
“m”>“n” and “m”≤5.

4. The pharmaceutical composition according to claim 3, wherein the at least one of the one or more layers further comprises at least one modification or termination “T” present on a surface of the layer body in which each “T” is selected from the group consisting of a hydroxyl group, a fluorine atom, a chlorine atom, an oxygen atom, and a hydrogen atom.

5. The pharmaceutical composition according to claim 1, wherein the MXene comprises Ti₃C₂.

6. The pharmaceutical composition according to claim 1, wherein the MXene is present in an amount effective to cause an increase in the amount or concentration of the one or more short-chain fatty acids in vivo in the human subject.

7. The pharmaceutical composition according to claim 1, wherein the intestinal bacterial flora comprise bacteria in class Clostridia of phylum Firmicutes, and the improvement comprises an increase in proliferation of the bacteria.

8. The pharmaceutical composition according to claim 1, wherein the intestinal bacterial flora comprise those in family Eggerthellaceae of order Coriobacteriales of class Coriobacteriia of phylum Actinobacteriota, those in family Ruminococcaceae of order Oscillospirales of class Clostridia of phylum Firmicutes, those in family Lachnospiraceae of order Lachnospirales of class Clostridia of phylum Firmicutes, or those in family Butyricicoccaceae of order Oscillospirales of class Clostridia of phylum Firmicutes.

9. The pharmaceutical composition according to claim 1, wherein the one or more short-chain fatty acids comprises butyric acid.

10. A method of treating a disease or condition in a human subject in need thereof, comprising
a) administering the pharmaceutical composition according to claim 1, to the subject, thereby increasing an amount or concentration of one or more short-chain fatty acids in the subject; and
b) reducing or eliminating one or more symptoms of the disease or condition;
wherein the administration results in (i) reparation of at least one blood vessel barrier and/or suppression of excessive enhancement of permeability, in the subject; (ii) a reduction in inflammation, in the subject; and/or (iii) a reduction in hypertension, in the subject.

11. The method according to claim 10, wherein the at least one blood vessel barrier is an intestinal barrier or a blood-brain barrier.

12. The method according to claim 11, wherein the administration results in a lowering of blood pressure, in the subject.

13. The pharmaceutical composition according to claim 1, wherein the pharmaceutical composition is formulated as an oral dosage form.

14. A method for improving intestinal bacterial flora or increasing a concentration or amount of at least one short-chain fatty acid in vivo, in a human subject in need thereof, comprising:
administering an effective amount of an MXene to the subject, thereof, thereby improving intestinal bacterial flora or increasing the concentration or amount of the at least one short-chain fatty acid in vivo, in the subject.

15. The method according to claim 14, wherein the MXene is administered orally.

16. A method for treating or preventing a disease, in a human subject in need thereof, the method comprising:
administering an effective amount of an MXene to the subject, thereby reducing or eliminating one or more symptoms of the disease, in the subject;
wherein an effective amount comprises an amount sufficient to result in reparation of at least one blood vessel barrier in the subject, and
wherein the disease comprises vascular dementia, Parkinson's disease, inflammatory bowel disease, chronic renal failure, irritable bowel syndrome, and/or ischemic and demyelinating central nervous system disease.

17. The method for treating or preventing a disease according to claim 16, wherein the MXene is administered orally.

18. The method for treating or preventing a disease according to claim 16, wherein the effective amount of the MXene comprises an amount within a range of 50 to 1,000 mg per day.

19. The method for treating or preventing a disease according to claim 18, wherein the one or more symptoms of the disease comprises reduced cognitive function and/or vascular dementia.

20. The method for treating or preventing a disease according to claim 18, wherein the one or more symptoms of the disease comprises hypertension.
